(19) United States
(12) Patent Application Publication
(10) Pub. No.: US 2023/0414727 A1
(43) Pub. Date: Dec. 28, 2023

(54) TISSUE KALLIKREIN-1 FOR TREATING CHRONIC KIDNEY DISEASE

(71) Applicant: DiaMedica Inc., Kelowna (CA)

(72) Inventors: Rick PAULS, Minnetonka, MN (US); David Wambeke, Edina, MN (US); Nick Paulson, St. Paul, MN (US)

(21) Appl. No.: 18/295,991

(22) Filed: Apr. 5, 2023

Related U.S. Application Data
(60) Provisional application No. 63/327,529, filed on Apr. 5, 2022.

(51) Int. Cl. A61K 38/48; A61P 13/12; C12Q 1/6883; C12Q 1/37

(52) U.S. Cl. CPC A61K 38/4853; A61P 13/12; C12Q 2600/156; C12Q 1/37; C12Y 304/21035; C12Q 1/6883

(57) ABSTRACT

Provided are methods of using human tissue kallikrein-1 (KLK1) to treat chronic kidney disease (CKD) in patients having salt-sensitive hypertension and low KLK1 levels, including methods of identifying and treating optimal sub-populations of CKD patients based on selected genotypes and/or phenotypes.

### CROSS-REFERENCE TO RELATED APPLICATIONS

This application claims the benefit under 35 U.S.C. § 119(e) of U.S. Provisional Application No. 63/327,529, filed Apr. 5, 2022, which is incorporated by reference in its entirety.

### STATEMENT REGARDING THE SEQUENCE LISTING

The Sequence Listing XML associated with this application is provided in XML file format and is hereby incorporated by reference into the specification. The name of the XML file containing the Sequence Listing XML is DIAM_042_01 US_ST26.xml. The XML file is about 5,456 bytes, was created on Apr. 4, 2023, and is being submitted electronically via USPTO Patent Center.

### BACKGROUND

### Technical Field

Embodiments of the present disclosure relate to methods of using human tissue kallikrein-1 (KLK1) to treat chronic kidney disease (CKD) in patients having low KLK1 levels and/or salt-sensitive hypertension, including methods of identifying and treating optimal sub-populations of CKD patients based on selected genotypes and/or phenotypes.

### Description of the Related Art

Tissue kallikreins all possess protease activity with a substrate specificity similar to that of trypsin or chymotrypsin. The most well-characterized activity of KLK1 is its enzymatic cleavage of kininogen to produce bradykinin (BK)-like peptides, collectively known as kinins, which activate, either directly or indirectly, subtypes of both bradykinin receptors (BK-B1, BK-B2). Activation of BK receptors by kinins set in motion a large number of complex metabolic pathways in response to ischemia within the body, which can include improved blood flow (through vasodilation), an anti-inflammatory response, cell repair through angiogenesis or vasculogenesis, and decrease of apoptosis.

Chronic kidney disease (CKD) is a type of kidney disease in which there is gradual loss of kidney function over a period of months to years. It is the cause of an increasing number of world-wide deaths (Wang et al., Lancet. 388 (10053): 1459-1544, 2016). There is a significant body of scientific studies which show that tissue kallikrein-mediated release increases blood flow in a variety of tissues including kidney (see, e.g., Stone et al., Arterioscler Thromb Vasc Biol. 29: 657-664, 2009), and that such is likely one mode by which kallikrein treatment addresses certain conditions. It is therefore believed that KLK1 has the potential to treat a broad spectrum of clinical scenarios, including where re-establishing blood flow and reducing inflammation in patients is vital to preserving kidney function.

However, there remains a need in the art to identify optimal sub-populations of CDK patients that will most benefit from KLK1 therapy.

### BRIEF SUMMARY

Embodiments of the present disclosure relate to methods of treating chronic kidney disease (CKD) in a patient in need thereof, comprising administering to the patient a pharmaceutical composition that comprises one or more tissue kallikrein (KLK1) polypeptides, wherein the patient has low KLK1 levels and/or salt-sensitive hypertension. In some embodiments, the low KLK1 levels are characterized by urinary KLK1 levels of about or less than about 15, 16, 17, 18, 19, 20, 25, 30, 35, or 40 ng/mL. Certain embodiments include the steps of determining KLK1 levels in a urine or blood/serum sample from the patient, and administering the pharmaceutical composition to the patient if urinary KLK1 levels are about or less than about 15, 16, 17, 18, 19, 20, 25, 30, 35, or 40 ng/mL.

In some embodiments, the patient has:
an R53H mutation in exon 3 of the KLK1 gene;
a 12G promoter allele in the KLK1 gene, which is characterized by 12 G repeats in the KLK1 gene locus starting at position −130 and ending at position −121;
an APOL1 gene mutation of the G1 haplotype, which is characterized by a terminal exon with two SNPs: rs73885319 and rs609101, and/or the G2 haplotype, which is characterized by a six base pair deletion: rs71785313;
a T594M mutation in the epithelial sodium-channel beta subunit (ENaC) gene;
a CYP11B1 gene mutation, characterized by an rs6410 single nucleotide polymorphism (SNP) and/or an rs6387 SNP;
a CYP11B2 gene mutation, which is characterized by an intron 2 conversion, an rs1799998 SNP, and/or an rs4539 SNP;
a SLC12A1 gene mutation, which is characterized by a base pair deletion, an insertion, and/or a nonconservative missense mutation in the SLC12A1 gene; and/or
a V142I mutation in the transthyretin (TTR) gene.

Certain embodiments include the steps of determining R53H mutation status in exon 3 of the KLK1 gene in a biological sample from the patient; and administering the pharmaceutical composition to the patient if the R53H mutation in exon 3 of the KLK1 gene is present in the biological sample, optionally if the R53H mutation is homozygous. Certain embodiments include the steps of determining 12G promoter allele status in the KLK1 gene in a biological sample from the patient; and administering the pharmaceutical composition to the patient if the 12G promoter allele in the KLK1 gene is present in the biological sample, optionally if the 12G promoter allele is homozygous.

Particular embodiments include the steps of determining APOL1 gene mutation status in a biological sample from the patient; and administering the pharmaceutical composition to the patient if the APOL1 gene mutation is present in the biological sample as at least one of the G1 haplotype and/or the G2 haplotype, optionally if the APOL1 gene mutation is homozygous. Some embodiments include the steps of determining T594M mutation status in the ENaC gene in a biological sample from the patient; and administering the pharmaceutical composition to the patient if the T594M mutation in the ENaC gene is present in the biological sample, optionally if the T594M mutation is homozygous.

Certain embodiments include the steps of determining CYP11B1 gene mutation status in a biological sample from the patient; and administering the pharmaceutical composition to the patient if the CYP11B1 gene mutation is present in the biological sample, optionally if the CYP11B1 gene mutation is homozygous. Some embodiments include the steps of determining CYP11B2 gene mutation status in a biological sample from the patient; and administering the pharmaceutical composition to the patient if the CYP11B2 gene mutation is present in the biological sample, optionally if the CYP11B2 gene mutation is homozygous.

Certain embodiments include the steps of determining SLC12A1 gene mutation status in a biological sample from the patient; and administering the pharmaceutical composition to the patient if the SLC12A1 gene mutation is present in the biological sample, optionally if the SLC12A1 gene mutation is homozygous. Certain embodiments include the steps of determining V142I mutation status in in the TTR gene in a biological sample from the patient; and administering the pharmaceutical composition to the patient if the V142I mutation in the TTR gene is present in the biological sample, optionally if the V142I mutation is homozygous.

Certain embodiments include determining mutation status or allele status in the biological sample by any one or more of DNA or RNA sequencing, polymerase chain reaction (PCR) optionally mutagenically separated PCR (MS-PCR), in situ hybridization (ISH), fluorescence in situ hybridization (FISH), whole exome sequencing (WES), single nucleotide polymorphism (SNP) array, next generation sequencing (NGS), or comparative genome hybridization (CGH) on the human gene.

In some embodiments, the patient is of African, Asian, Spanish, or Polynesian descent, optionally an African-American. In some embodiments, the patient has sickle cell disease and/or focal segmental glomerulosclerosis (FSGS).

Certain embodiments comprise obtaining the biological sample from the patient. In some embodiments, the biological sample is a blood/serum sample or a urine sample.

In some embodiments, the pharmaceutical composition comprises DM199.

In some embodiments, the pharmaceutical composition comprises a first KLK1 polypeptide and a second KLK1 polypeptide, wherein the first KLK1 polypeptide has three glycans attached at three different positions per polypeptide and the second KLK1 polypeptide has two glycans attached at two different positions per polypeptide; and wherein the first KLK1 polypeptide and the second KLK1 polypeptides are present in the pharmaceutical composition at a ratio of about 45:55 to about 55:45. In some embodiments, one or more of the glycans are N-linked glycans. In some embodiments, one or more of the glycans are attached at amino acid residues 78, 84, or 141 of KLK1 as defined by SEQ ID NO: 3 or 4. In some embodiments, the three glycans of the first KLK1 polypeptide are N-linked glycans at residues 78, 84, and 141. In some embodiments, the two glycans of the second KLK1 polypeptide are N-linked glycans at residues 78 and 84 but not 141. In some embodiments, the first KLK1 polypeptide and the second KLK1 polypeptides are present in the pharmaceutical composition at a ratio of about 50:50.

In some embodiments, the one or more KLK1 polypeptide(s) are recombinant KLK polypeptides, mature KLK1 polypeptides, human KLK1 (hKLK1) polypeptides, or any combination thereof. In some embodiments, the hKLK1 polypeptide(s) comprise, consist, or consist essentially of amino acid residues 78-141 of SEQ ID NO:1 or amino acids residues 78-141 SEQ ID NO:2, or an active fragment thereof, or an active variant having at least about 90, 95, 96, 97, 98, or 99% sequence identity to amino acid residues 78-141 of SEQ ID NO:1 or amino acids residues 78-141 SEQ ID NO:2. In some embodiments, the hKLK1 polypeptide(s) comprise, consist, or consist essentially of amino acid residues 25-262 of SEQ ID NO:1 or amino acid residues 25-262 of SEQ ID NO:2, or an active fragment thereof, or an active variant having at least about 90, 95, 96, 97, 98, or 99% sequence identity to amino acid residues 25-262 of SEQ ID NO:1 or amino acid residues 25-262 of SEQ ID NO:2.

In some embodiments, the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises E145 and/or A188. In some embodiments, the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises Q145 and/or V188.

In some embodiments, the pharmaceutical composition is formulated at a total KLK1 polypeptide dosage of about 0.5 μg/kg to about 10.0 μg/kg. In some embodiments, the pharmaceutical composition is formulated at a total KLK1 polypeptide dosage of about 2 μg/kg or about 4 μg/kg, or about 0.1, 0.2, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 μg/kg. In some embodiments, the pharmaceutical composition is formulated at a total KLK1 polypeptide dosage of about 1.0 μg/kg to about 5.0 μg/kg, or about 1.0 μg/kg to about 4.0 μg/kg, or about 1.0 μg/kg to about 3.0 μg/kg, or about 1.0 μg/kg to about 2.0 μg/kg, or about 2.0 μg/kg to about 5.0 μg/kg, or about 2.0 μg/kg to about 4.0 μg/kg, or about 2.0 μg/kg to about 3.0 μg/kg, or about 3.0 μg/kg to about 5.0 μg/kg, or about 3.0 μg/kg to about 4.0 μg/kg, or about 4.0 μg/kg to about 5.0 μg/kg.

Certain embodiments comprise subcutaneously or intravenously administering the pharmaceutical composition to the patient. In some embodiments, the pharmaceutical composition comprises a pharmaceutically acceptable excipient, diluent, adjuvant, or carrier. In some embodiments, the pharmaceutical composition is substantially free of other glycosylated isoforms (glycoforms) of KLK1. In some embodiments, the pharmaceutical composition has endotoxin levels of less than about 1 EU/mg protein, host cell protein of less than about 100 ng/mg total protein, host cell DNA of less than about 10 pg/mg total protein, and/or is substantially free of aggregates (greater than about 95% appearing as a single peak by SEC HPLC).

In some embodiments, administering the pharmaceutical composition improves one or more clinical parameters in the patient. In some embodiments, the one or more clinical parameters are selected from decreased albuminuria (UACR), increased estimated glomerular filtration rate (eGFR), decreased blood pressure, serum KLK1 levels of about 1-5 ng/ml, decreased swelling, optionally in the lower extremities of the patient, and decreased risk of cardiovascular events in the patient, optionally myocardial infarction or stroke. In some embodiments, administering the pharmaceutical composition decreases UACR by about or at least about 25, 30, 35, 40, 45, 50, 55, 60, 65, or 70% or more.

### BRIEF DESCRIPTION OF THE DRAWINGS

FIG. 1 shows the statistically significant differences in urinary KLK levels between healthy subjects and patients with chronic kidney disease (CKD).

### DETAILED DESCRIPTION

### Defintions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e., to at least one) of the grammatical object of the article. By way of example, “an element” means one element or more than one element.

By “about” is meant a quantity, level, value, number, frequency, percentage, dimension, size, amount, weight or length that varies by as much as 30, 25, 20, 15, 10, 9, 8, 7, 6, 5, 4, 3, 2 or 1% to a reference quantity, level, value, number, frequency, percentage, dimension, size, amount, weight or length.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they materially affect the activity or action of the listed elements.

As used herein, the term “amino acid” is intended to mean both naturally occurring and non-naturally occurring amino acids as well as amino acid analogs and mimetics. Naturally-occurring amino acids include the 20 (L)-amino acids utilized during protein biosynthesis as well as others such as 4-hydroxyproline, hydroxylysine, desmosine, isodesmosine, homocysteine, citrulline and ornithine, for example. Non-naturally occurring amino acids include, for example, (D)-amino acids, norleucine, norvaline, p-fluorophenylalanine, ethionine and the like, which are known to a person skilled in the art. Amino acid analogs include modified forms of naturally and non-naturally occurring amino acids. Such modifications can include, for example, substitution or replacement of chemical groups and moieties on the amino acid or by derivatization of the amino acid. Amino acid mimetics include, for example, organic structures which exhibit functionally similar properties such as charge and charge spacing characteristic of the reference amino acid. For example, an organic structure which mimics arginine (Arg or R) would have a positive charge moiety located in similar molecular space and having the same degree of mobility as the e-amino group of the side chain of the naturally occurring Arg amino acid. Mimetics also include constrained structures so as to maintain optimal spacing and charge interactions of the amino acid or of the amino acid functional groups. Those skilled in the art know or can determine what structures constitute functionally equivalent amino acid analogs and amino acid mimetics.

The terms “endotoxin free” or “substantially endotoxin free” relate generally to dosage forms, compositions, solvents, devices, and/or vessels that contain at most trace amounts (e.g., amounts having no clinically adverse physiological effects to a subject) of endotoxin, and preferably undetectable amounts of endotoxin. Endotoxins are toxins associated with certain bacteria, typically gram-negative bacteria, although endotoxins may be found in gram-positive bacteria, such as Listeria monocytogenes. The most prevalent endotoxins are lipopolysaccharides (LPS) or lipo-oligo-saccharides (LOS) found in the outer membrane of various Gram-negative bacteria, and which represent a central pathogenic feature in the ability of these bacteria to cause disease. Small amounts of endotoxin in humans may produce fever, a lowering of the blood pressure, and activation of inflammation and coagulation, among other adverse physiological effects.

Therefore, in pharmaceutical production, it is often desirable to remove most or all traces of endotoxin from drug products and/or drug containers, because even small amounts may cause adverse effects in humans. A depyrogenation oven may be used for this purpose, as temperatures in excess of 300° C. are typically required to break down most endotoxins. For instance, based on primary packaging material such as syringes or vials, the combination of a glass temperature of 250° C. and a holding time of 30 minutes is often sufficient to achieve a 3 log reduction in endotoxin levels. Other methods of removing endotoxins are contemplated, including, for example, chromatography and filtration methods, as described herein and known in the art. Also included are methods of producing KLK1 polypeptides in and isolating them from eukaryotic cells such as mammalian cells to reduce, if not eliminate, the risk of endotoxins being present in a composition of the invention. Preferred are methods of producing KLK1 polypeptides in and isolating them from recombinant cells grown in chemically defined, serum free media.

Endotoxins can be detected using routine techniques known in the art. For example, the Limulus Amebocyte Lysate assay, which utilizes blood from the horseshoe crab, is a very sensitive assay for detecting presence of endotoxin. In this test, very low levels of LPS can cause detectable coagulation of the limulus lysate due a powerful enzymatic cascade that amplifies this reaction. Endotoxins can also be quantitated by enzyme-linked immunosorbent assay (ELISA). To be substantially endotoxin free, endotoxin levels may be less than about 0.001, 0.005, 0.01, 0.02, 0.03, 0.04, 0.05, 0.06, 0.08, 0.09, 0.1, 0.5, 1.0, 1.5, 2, 2.5, 3, 4, 5, 6, 7, 8, 9, or 10 EU/ml, or EU/mg protein. Typically, 1 ng lipopolysaccharide (LPS) corresponds to about 1-10 EU.

The “half-life” of an agent such as a KLK1 polypeptide (e.g., DM199) can refer to the time it takes for the agent to lose half of its pharmacologic, physiologic, or other activity, relative to such activity at the time of administration into the serum or tissue of an organism, or relative to any other defined time-point. “Half-life” can also refer to the time it takes for the levels of agent to be reduced by half of a starting amount administered into the serum or tissue of an organism, relative to such amount or concentration at the time of administration into the serum or tissue of an organism, or relative to any other defined time-point. The half-life can be measured in serum and/or any one or more selected tissues.

The terms “modulating” and “altering” include “increasing,” “enhancing” or “stimulating,” as well as “decreasing” or “reducing,” typically in a statistically significant or a physiologically significant amount or degree relative to a control. An “increased,” “stimulated” or “enhanced” amount is typically a “statistically significant” amount, and may include an increase that is 1.1, 1.2, 2, 3, 4, 5, 6, 7, 8, 9, 10, 15, 20, 30 or more times (e.g., 500, 1000 times) (including all integers and decimal points in between and above 1, e.g., 1.5, 1.6, 1.7. 1.8, etc.) the amount or level produced by a control composition, sample or test subject. A “decreased” or “reduced” amount is typically a “statistically significant” amount, and may include a 1%, 2%, 3%, 4%, 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 65%, 70%, 75%, 80%, 85%, 90%, 95%, or 100% decrease in the amount or level produced a control composition, sample or test subject. Examples of comparisons and “statistically significant” amounts are described herein.

The terms “polypeptide,” “protein” and “peptide” are used interchangeably and mean a polymer of amino acids not limited to any particular length. The term “enzyme” includes polypeptide or protein catalysts. The terms include modifications such as myristoylation, sulfation, glycosylation, phosphorylation and addition or deletion of signal sequences. The terms “polypeptide” or “protein” means one or more chains of amino acids, wherein each chain comprises amino acids covalently linked by peptide bonds, and wherein said polypeptide or protein can comprise a plurality of chains non-covalently and/or covalently linked together by peptide bonds, having the sequence of native proteins, that is, proteins produced by naturally-occurring and specifically non-recombinant cells, or genetically-engineered or recombinant cells, and comprise molecules having the amino acid sequence of the native protein, or molecules having deletions from, additions to, and/or substitutions of one or more amino acids of the native sequence. In certain embodiments, the polypeptide is a “recombinant” polypeptide, produced by recombinant cell that comprises one or more recombinant DNA molecules, which are typically made of heterologous polynucleotide sequences or combinations of polynucleotide sequences that would not otherwise be found in the cell.

The term “reference sequence” refers generally to a nucleic acid coding sequence, or amino acid sequence, to which another sequence is being compared. All polypeptide and polynucleotide sequences described herein are included as references sequences, including those described by name and those described in the Tables and the Sequence Listing.

A result is typically referred to as “statistically significant” if it is unlikely to have occurred by chance. The significance level of a test or result relates traditionally to the amount of evidence required to accept that an event is unlikely to have arisen by chance. In certain cases, statistical significance may be defined as the probability of making a decision to reject the null hypothesis when the null hypothesis is actually true (a decision known as a Type I error, or “false positive determination”). This decision is often made using the p-value: if the p-value is less than the significance level, then the null hypothesis is rejected. The smaller the p-value, the more significant the result. Bayes factors may also be utilized to determine statistical significance (see Goodman, Ann Intern Med. 130:1005-13, 1999).

The term “solubility” refers to the property of a KLK1 polypeptide provided herein to dissolve in a liquid solvent and form a homogeneous solution. Solubility is typically expressed as a concentration, either by mass of solute per unit volume of solvent (g of solute per kg of solvent, g per dL (100 mL), mg/ml, etc.), molarity, molality, mole fraction or other similar descriptions of concentration. The maximum equilibrium amount of solute that can dissolve per amount of solvent is the solubility of that solute in that solvent under the specified conditions, including temperature, pressure, pH, and the nature of the solvent. In certain embodiments, solubility is measured at physiological pH, or other pH, for example, at pH 6.0, pH 7.0, pH 7.4, pH 8.0 or pH 9.0. In certain embodiments, solubility is measured in water or a physiological buffer such as PBS or NaCl (with or without NaP). In specific embodiments, solubility is measured at relatively lower pH (for example, pH 6.0) and relatively higher salt (for example, 500 mM NaCl and 10 mM NaP). In certain embodiments, solubility is measured in a biological fluid (solvent) such as blood or serum. In certain embodiments, the temperature can be about room temperature (for example, about 20, about 21, about 22, about 23, about 24, or about 25° C.) or about body temperature (37° C.). In certain embodiments, a KLK1 polypeptide has a solubility of at least about 1, at least about 2, at least about 3, at least about 4, at least about 5, at least about 6, at least about 7, at least about 8, at least about 9, at least about 10, at least about 11, at least about 12, at least about 13, at least about 14, at least about 15, at least about 16, at least about 17, at least about 18, at least about 19, at least about 20, at least about 25, at least about 30, at least about 35, at least about 40, at least about 45, at least about 50, or at least about 60 mg/ml at room temperature or at 37° C.

“Substantially” or “essentially” means nearly totally or completely, for instance, 95%, 96%, 97%, 98%, 99% or greater of some given quantity.

“Treatment” or “treating,” as used herein, includes any desirable effect on the symptoms or pathology of a disease or condition, and may include even minimal changes or improvements in one or more measurable markers of the disease or condition being treated. “Treatment” or “treating” does not necessarily indicate complete eradication or cure of the disease or condition, or associated symptoms thereof. The subject receiving this treatment is any subject in need thereof. Exemplary markers of clinical improvement will be apparent to persons skilled in the art.

As used herein, the terms “therapeutically effective amount”, “therapeutic dose,” “prophylactically effective amount,” or “diagnostically effective amount” is the amount of an agent such as a KLK1 polypeptide (e.g., DM199) needed to elicit the desired biological response following administration.

A “subject,” as used herein, includes any animal that exhibits a symptom, or is at risk for exhibiting a symptom, which can be treated with a KLK1 polypeptide or a dosage form thereof. Suitable subjects (patients) include laboratory animals (such as mouse, rat, rabbit, or guinea pig), farm animals, and domestic animals or pets (such as a cat or dog). Non-human primates and, preferably, human patients, are included.

By “isolated” is meant material that is substantially or essentially free from components that normally accompany it in its native state. For example, an “isolated peptide” or an “isolated polypeptide” and the like, as used herein, includes the in vitro isolation and/or purification of a peptide or polypeptide molecule from its natural cellular environment, and from association with other components of the cell; i.e., it is not significantly associated with in vivo substances such as host cell proteins or nucleic acids.

A “wild type” or “reference” sequence or the sequence of a “wild type” or “reference” protein/polypeptide may be the reference sequence from which variant polypeptides are derived through the introduction of changes. In general, the “wild type” amino acid sequence for a given protein is the sequence that is most common in nature. Similarly, a “wild type” gene sequence is the polynucleotide sequence for that gene which is most commonly found in nature. Mutations can be introduced into a “wild type” gene (and thus the protein it encodes) either through natural processes or through human induced means.

Each embodiment in this specification is to be applied to every other embodiment unless expressly stated otherwise.

Embodiments of the present disclosure relate to methods of treating chronic kidney disease (CKD) in a patient of African, Asian, Spanish, or Polynesian descent in need thereof, comprising administering to the patient a pharmaceutical composition that comprises one or more tissue kallikrein (KLK1) polypeptides, wherein the patient has low KLK1 levels and/or salt-sensitive hypertension. Hypertension (HTN or HT), also known as high blood pressure (HBP), is a condition in which the blood pressure in the arteries is persistently elevated. Blood pressure is classified by two measurements, the systolic and diastolic pressures, which are the maximum and minimum pressures, respectively. Healthy adults at rest typically have a systolic blood pressure in the range of about 100-130 millimeters mercury (mmHg), and a diastolic blood pressure in the range of about 60-80 mmHg diastolic.

In some embodiments, a patient with “salt-sensitive hypertension” demonstrates meaningful or statistically significant increases in blood pressure (e.g., ≥3, 4, 5 mmHg) in response to increased dietary salt intake, mainly sodium salts such as sodium chloride (NaCl), or vice versa, that is, meaningful decreases (e.g., ≥3, 4, 5 mmHg) in blood pressure in response to reduced dietary salt intake. One exemplary definition of “salt-sensitive hypertension” is an increase in mean arterial blood pressure (MAP) of at least 4 mmHg during 24-hour ambulatory blood pressure monitoring (ABPM) with an increase in NaCl intake. Salt-sensitive hypertension can be diagnosed or identified using routine techniques in the art, for example, by monitoring for significantly greater blood pressure changes at the end of a high-salt diet (e.g., over 1 week) than at the end of a low-salt diet (e.g., over 1 additional week), or from 24-hour ABPM data, genetic screening, cell-based assays, and the use of urine exosomes as markers (see, for example, Castiglioni et al., Hypertension. 57:180-185, 2011; and Felder et al., Curr Opin Nephrol Hypertens. 22:65-76, 2013). In certain embodiments, a patient with salt-sensitive hypertension has a systolic blood pressure of about or at least about 130, 135, 140, 145, 150, 155, 160 or higher, and a diastolic blood pressure of about or at least about 80, 85, 90, 95, or 100 or higher.

In certain embodiments, the patient in need thereof has low KLK1 levels (see, for example, Song et al., J. Human Hypertension. 14:461-468, 2000; and Naicker et al., Immunopharm. 44:183-192, 1999). In some embodiments, the low KLK1 levels are characterized by urinary KLK1 levels of about or less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 ng/mL (e.g., about or less than about the 50^{th} 40^{th} 30^{th} 25^{th} or 20^{th} percentile, as per the values in Table E1). In specific embodiments, the low KLK1 levels are characterized by urinary KLK1 levels of about or less than about 39 or 40 ng/mL (50^{th} percentile).

Certain embodiments include the step of determining KLK1 levels/activity in a urine or blood/serum sample from the patient, and administering a pharmaceutical composition of KLK1 to the patient if urinary KLK1 levels are about or less than about 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 ng/mL (e.g., about or less than about the 50^{th} 40th 30th 25th or 20^{th}, percentile, as per the values in Table E1). Specific embodiments include the step of determining KLK1 levels/activity in a urine or blood/serum sample from the patient, and administering a pharmaceutical composition of KLK1 to the patient if urinary KLK1 levels are about or less than about 39 or 40 ng/mL (50^{th} percentile).

In certain embodiments, the patient in need thereof has or is characterized by having one or more specific genotypes. Certain embodiments include the step of selecting the patient for KLK1 therapy based on identifying or determining the genotype status of the patient in need thereof, and administering a pharmaceutical composition of KLK1 to the patient if the specific genotype is present in the patient. Examples of genotypes that can be used to select a patient for KLK1 therapy include the following:
an R53H mutation in exon 3 of the KLK1 gene;
a 12G promoter allele in the KLK1 gene, which is characterized by 12 G repeats in the KLK1 gene locus starting at position −130 and ending at position −121;
an APOL1 gene mutation of the G1 haplotype, which is characterized by a terminal exon with two SNPs: rs73885319 and rs609101, and/or the G2 haplotype, which is characterized by a six base pair deletion: rs71785313;
a T594M mutation in the epithelial sodium-channel beta subunit (ENaC) gene;
a CYP11B1 gene mutation, characterized by an rs6410 single nucleotide polymorphism (SNP) and/or an rs6387 SNP;
a CYP11B2 gene mutation, which is characterized by an intron 2 conversion, an rs1799998 SNP, and/or an rs4539 SNP;
a SLC12A1 gene mutation, which is characterized by a base pair deletion, an insertion, and/or a nonconservative missense mutation in the SLC12A1 gene; and/or
a V142I mutation in the transthyretin (TTR) gene.

In some embodiments, the patient in need thereof has an R53H mutation in exon 3 of the KLK1 gene. The R53H mutation is a missense polymorphism, which results in the insertion of a histidine for arginine at position 53 in human tissue (urinary) KLK (R53H) and is associated with the loss of kinin-generating activity (see, for example, Blanchard et al., Clin J Am Soc Nephrol 2:320-325, 2007; Slim et al., J. Am. Soc. Nephrol. 13: 968-976, 2002; and NM_002257.4 (KLK1):c.230G>A (p.Arg77His). In some instances, the patient is heterozygous for the R53H mutation. In some instances, the patient is homozygous for the R53H mutation. Some embodiments include the steps of determining R53H mutation status in exon 3 of the KLK1 gene in a biological sample from the patient; and administering the pharmaceutical composition to the patient if the R53H mutation in exon 3 of the KLK1 gene is present in the biological sample, including if the R53H mutation is homozygous or heterozygous.

In some embodiments, the patient in need thereof has a 12G promoter allele in the KLK1 gene, which is characterized by 12 G repeats in the KLK1 gene locus starting at about position −130 and ending at about position −121 (see, for example, Yu et al., Kidney International. 61:1030-1039, 2002). In some instances, the patient is heterozygous for the 12G promoter allele. In some instances, the patient is homozygous for the 12G promoter allele. Some embodiments include the steps of determining 12G promoter allele status in the KLK1 gene in a biological sample from the patient; and administering the pharmaceutical composition to the patient if the 12G promoter allele in the KLK1 gene is present in the biological sample, including if the 12G promoter allele is homozygous or heterozygous.

In some embodiments, the patient in need thereof has an APOL1 gene mutation (see, for example, Reidy et al., Curr Opin Pediatr. 30: 252-259, 2018). Examples include an APOL1 gene mutation of the G1 haplotype, which is characterized by a terminal exon with two SNPs: rs73885319 and rs609101. Also included is an APOL1 gene mutation of the G2 haplotype, which is characterized by a six base pair deletion: rs71785313. In some embodiments, the patient is heterozygous for the APOL1 gene mutation. In some embodiments, the patient is homozygous for the APOL1 gene mutation. Some embodiments include the steps of determining APOL1 gene mutation status in a biological sample from the patient; and administering the pharmaceutical composition to the patient if the APOL1 gene mutation is present in the biological sample as at least one of the G1 haplotype and/or the G2 haplotype, including if the APOL1 gene mutation is homozygous or heterozygous.

In some embodiments, the patient in need thereof has a T594M mutation in the epithelial sodium-channel beta subunit (ENaC) gene (see, for example, Pratt, J. Am. Soc. Nephrology 16:3154-3159, 2005; and Pratt et al., Hypertension 40:903-908, 2002). In some instances, the patient is heterozygous for the T594M mutation. In some instances, the patient is homozygous for the T594M mutation. Some embodiments include the steps of determining T594M mutation status in the ENaC gene in a biological sample from the patient; and administering the pharmaceutical composition to the patient if the T594M mutation in the ENaC gene is present in the biological sample, including if the T594M mutation is homozygous or heterozygous.

In certain embodiments, the patient in need thereof has a CYP11B1 gene mutation, a CYP11B2 gene mutation, or both (see, for example, Zhang et al., Hypertension Res. 33: 478-484, 2010). Examples include a CYP11B1 gene mutation that is characterized by an intron 2 conversion, an rs1799998 SNP, and/or an rs4539 SNP. In some instances, the patient is heterozygous for the CYP11B1 gene mutation. In some instances, the patient is homozygous for the CYP11B1 gene mutation. Some embodiments include the steps of determining CYP11B1 gene mutation status in a biological sample from the patient; and administering the pharmaceutical composition to the patient if the CYP11B1 gene mutation is present in the biological sample, including if the CYP11B1 gene mutation is homozygous or heterozygous. Also included is a CYP11B2 gene mutation that is characterized by an intron 2 conversion, an rs1799998 SNP, and/or an rs4539 SNP. In some instances, the patient is heterozygous for the CYP11B2 gene mutation. In some instances, the patient is homozygous for the CYP11B2 gene mutation. Some embodiments include the steps of determining CYP11B2 gene mutation status in a biological sample from the patient; and administering the pharmaceutical composition to the patient if the CYP11B2 gene mutation is present in the biological sample, including if the CYP11B2 gene mutation is homozygous or heterozygous.

In certain embodiments, the patient in need thereof has a SLC12A1 gene mutation; the SLC12A1 encodes the NKCC2 isoform of the Na—K—Cl cotransporter (see, for example, Simon et al., Nat Genet. 13: 183-8, 1996). Examples include a base pair deletion, an insertion, and/or a nonconservative missense mutation in the SLC12A1 gene (supra). In some instances, the patient is heterozygous for the SLC12A1 gene mutation. In some instances, the patient is homozygous for the SLC12A1 gene mutation. Particular embodiments include the steps of determining SLC12A1 gene mutation status in a biological sample from the patient, and administering the pharmaceutical composition to the patient if the SLC12A1 gene mutation is present in the biological sample, including if the SLC12A1 gene mutation is homozygous or heterozygous.

In certain embodiments, the patient in need thereof has a V142I mutation in the transthyretin (TTR) gene (see, for example, Coniglio et al., JACC Heart Fail. 10: 129-138, 2022). In some instances, the patient is heterozygous for the V142I mutation. In some instances, the patient is homozygous for the V142I mutation. Some embodiments include the steps of determining V142I mutation status in in the TTR gene in a biological sample from the patient, and administering the pharmaceutical composition to the patient if the V142I mutation in the TTR gene is present in the biological sample, including if the V142I mutation is homozygous or heterozygous.

Methods for determining mutation status or allele status in the biological sample are known in the art. Examples including methods of determining mutation or allele status by any one or more of DNA or RNA sequencing, polymerase chain reaction (PCR) including mutagenically separated PCR (MS-PCR; see, for example, Rust et al., Nucleic Acids Res. 21:3623-9, 1993), in situ hybridization (ISH), fluorescence in situ hybridization (FISH), whole exome sequencing (WES), single nucleotide polymorphism (SNP) array, next generation sequencing (NGS), or comparative genome hybridization (CGH) on the human gene of interest.

As noted above, in certain embodiments the patient in need thereof (with salt-sensitive hypertension and low KLK1 levels) is of African, Asian, Spanish, or Polynesian descent, for example, wherein the patient is African-America (see, for example, Ferdinand and Ferdinand, Expert Rev Cardiovasc Ther. 10(6):1357-1366, 2008). In certain embodiments, the patient of African descent is from at least one ancestral cluster (see, for example, Tishkoff et al., Science. 324 (5930): 1037-39, 2009; and Schlebusch and Jakobsson, Annu Rev Genomics Hum Genet. 19:405-428, 2018), including the Afroasiatic-speaking populations in or from North Africa and Northeast Africa; the Nilo-Saharan-speaking populations from Northeast Africa and East Africa; the Ari populations in or from Northeast Africa; the Niger-Congo-speaking populations in or from West-Central Africa, West Africa, East Africa, and Southern Africa; the Pygmy populations in or from Central Africa; and the Khoisan populations in or from Southern Africa. In some embodiments, the patient is African-American, for example, the African-American descendants of the West and Central Africans.

In certain embodiments, the patient is of Asian descent, for example, Central Asian descent (for example, Kazakhstan, Kyrgyzstan, Tajikistan, Uzbekistan, Turkmenistan, Xinjiang of western China, Mongolia, northern Pakistan), East Asian descent (for example, China, Hong Kong, Macau, Taiwan, Japan, Mongolia, North Korea, South Korea; including East Asians from the Han, Korean, Yamato, Bai, Hui, Tibetans, Turkic, Manchus, Ryukyuan, Ainu, Zhuang, and Mongol ethnic groups), South Asian descent (for example, Afghanistan, Bangladesh, Bhutan, India, Maldives, Nepal, Pakistan, Sri Lanka), or Southeast Asian descent (for example, Burma, Cambodia, Laos, Peninsular Malaysia, Thailand, Vietnam, Brunei, East Timor, Indonesia, East Malaysia, the Philippines, Singapore).

In some embodiments, the patient is of Polynesian descent, which refers to an ethnolinguistic group of closely related people who are native to Polynesia (islands in the Polynesian Triangle), an expansive region of Oceania in the Pacific Ocean (for example, Polynesian nation-states (Samoa, Niue, Cook Islands, Tonga, and Tuvalu) or form minorities in countries such as Australia, Chile (Easter Island), New Zealand, France (French Polynesia and Wallis and Futuna), and the United States (Hawaii and American Samoa), in addition to the British Overseas Territory of the Pitcairn Islands. Examples include Samoans, Tongans, Niueans, Cook Islands Maori, Tahitian Mā'ohi, Hawaiian Māol, Marquesans, and New Zealand Maori.

In some embodiments, the patient has sickle cell disease. Sickle cell disease refers to a group of inherited blood disorders, including sickle cell anemia. Sickle cell disease has an autosomal recessive pattern of inheritance, and in patients with sickle cell disease, at least one or both of the β-globin subunits in haemoglobin A is replaced with “haemoglobin S”—a single nucleotide polymorphism (SNP; GAG codon changing to GTG) of the β-globin gene, which results in glutamate being substituted by valine at position 6 (E6V substitution).

In some embodiments, the patient has focal segmental glomerulosclerosis (FSGS), also known as “focal glomerular sclerosis” or “focal nodular glomerulosclerosis,” FSGS refers to a histopathologic finding of scarring (sclerosis) of glomeruli and damage to renal podocytes (see, for example, Rosenberg et al., Clinical Journal of the American Society of Nephrology. 12: 502-517, 2017). This process damages the filtration function of the kidney, resulting in protein loss in the urine. Signs and symptoms include proteinuria, water retention, and edema (see, for example, Rydel et al., Am J Kidney Dis. 25: 534-42, 1995). Kidney failure is a common long-term complication of disease (see, for example, Korbet et al., Am J Kidney Dis. 23: 773-83, 1994).

Certain embodiments comprise the step of obtaining the biological sample from the patient. In some embodiments, the biological sample is a blood/serum sample or a urine sample.

Tissue Kallikrein-1 (KLK1) Polypeptides. As noted herein, certain methods, pharmaceutical compositions, or dosage forms comprise one or more tissue kallikrein-1 or KLK1 polypeptides. Tissue kallikreins are members of a gene super family of serine proteases comprising at least 15 separate and distinct proteins (named tissue kallikrein 1 through 15) (Yousef et al., 2001, Endocrine Rev; 22:184-204). Tissue kallikrein-1 is a trypsin-like serine protease. In humans and animal tissues, tissue kallikrein-1 cleaves kininogen into lysyl-bradykinin (also known as kallidin), a decapeptide kinin having physiologic effects similar to those of bradykinin. Bradykinin is a peptide that causes blood vessels to dilate and therefore causes blood pressure to lower. Kallidin is identical to bradykinin with an additional lysine residue added at the N-terminal end and signals through the bradykinin receptor.

The KLK1 gene encodes a single pre-pro-enzyme that is 262 amino acid residues in length and that includes the “pre-” sequence (residues 1-18) and the “pro-” sequence (residues 19-24), which is activated by trypsin-like enzymes. The “mature” and “active” form human KLK1 is a glycoprotein of about 238 amino acid residues (residues 25-262) with a molecular weight of 26 kDa and a theoretical pI of 4.6. KLK1 has five disulfide bonds in its tertiary structure that are believed to be responsible for the protein's high stability, both against trypsin digestion and heat inactivation.

The amino acid sequence of full-length tissue kallikrein-1 is available for a wide variety of species, including, but not limited to, human (SEQ ID NO:1 and SEQ ID NO:2), mouse (see, for example, GenBank: AAA39349.1, Feb. 1, 1994); domestic cat (see, for example, NCBI Reference Sequence: XP_003997527.1, Nov. 6, 2012); gorilla (see, for example, NCBI Reference Sequence: XP_004061305.1, Dec. 3, 2012); cattle (see, for example, GenBank: AAI51559.1, Aug. 2, 2007); dog (see, for example, CBI Reference Sequence: NP_001003262.1, Feb. 22, 2013); rat (see, for example, GenBank: CAE51906.1, Apr. 25, 2006); and olive baboon (see, for example, NCBI Reference Sequence: XP_003916022.1, Sep. 4, 2012). KLK1 is functionally conserved across species in its capacity to release the vasoactive peptide, Lys-bradykinin, from low molecular weight kininogen. A tissue kallikrein-1 polypeptide of the present invention may have any of the known amino acid sequences for KLK1, or a fragment or variant thereof.

In certain embodiments, the KLK1 polypeptide is a “mature” KLK1 polypeptide. In certain embodiments, the KLK1 polypeptide is a human KLK1 polypeptide, optionally a mature human KLK1 polypeptide. In particular embodiments, the KLK1 polypeptide is a recombinant human polypeptide, for example, a recombinant human KLK1 polypeptide, optionally in the mature form. Recombinant human KLK1 (rhKLK1) can provide certain advantages over other sources of KLK1, such as urinary KLK1 (e.g., human KLK1 isolated from human urine), including a homogenous preparation of rhKLK1, simpler regulatory path to licensure, and options to alter the amino acid sequence or glycosylation pattern based on cell culture conditions.

Exemplary amino acid sequences of human tissue kallikrein-1 (hKLK1) polypeptides are provided in Table K1 below.

TABLE K1
Exemplary KLK1 Sequences

| | | SEQ |
|---|---|---|
| | | ID |
| Source | Sequence | NO: |
| Human | MWFLVLCLALSLGGTGAAPPIQSRIVGGWE | 1 |
| KLK1 | CEQHSQPWQAALYHFSTFQCGGILVHROWV | |
| | LTAAHCISDNYQLWLGRHNLFDDENTAQFV | |
| | HVSESFPHPGFNMSLLENHTRQADEDYSHD | |
| | LMLLRLTEPADTITDAVKVVELPTEEPEVG | |
| | STCLASGWGSIEPENFSFPDDLQCVDLKIL | |
| | PNDECKKAHVQKVTDFMLCVGHLEGGKDTC | |
| | VGDSGGPLMCDGVLQGVTSWGYVPCGTPNK | |
| | PSVAVRVLSYVKWIEDTIAENS | |
| Human | MWFLVLCLALSLGGTGAAPPIQSRIVGGWE | 2 |
| KLK1 | CEQHSQPWQAALYHFSTFQCGGILVHROWV | |
| variant | LTAAHCISDNYQLWLGRHNLFDDENTAQFV | |
| | HVSESFPHPGENMSLLENHTRQADEDYSHD | |
| | LMLLRLTEPADTITDAVKVVELPTQEPEVG | |
| | STCLASGWGSIEPENFSFPDDLQCVDLKIL | |
| | PNDECKKVHVQKVTDFMLCVGHLEGGKDTC | |
| | VGDSGGPLMCDGVLQGVTSWGYVPCGTPNK | |
| | PSVAVRVLSYVKWIEDTIAENS | |
| Human | ivggweceqhsqpwqaalyhfstfqcggil | 3 |
| mature | vhrqwvltaahcisdnyqlwlgrhnlfdde | |
| KLK1 | ntaqfvhvsesfphpgfnmsllenhtrqad | |
| | edyshdlmllrltepadtitdavkvvelpt | |
| | eepevgstclasgwgsiepenfsfpddlqc | |
| | vdlkilpndeckkahvqkvtdfmlcvghle | |
| | ggkdtcvgdsggplmcdgvlqgvtswgyvp | |
| | cgtpnkpsvavrvlsyvkwiedtiaens | |
| Human | IVGGWECEQHSQPWQAALYHFSTFQCGGIL | 4 |
| mature | VHRQWVLTAAHCISDNYQLWLGRHNLFDDE | |
| KLK1 | NTAQFVHVSESFPHPGFNMSLLENHTRQAD | |
| variant | EDYSHDLMLLRLTEPADTITDAVKVVELPT | |
| | QEPEVGSTCLASGWGSIEPENFSFPDDLQC | |
| | VDLKILPNDECKKVHVQKVTDFMLCVGHLE | |
| | GGKDTCVGDSGGPLMCDGVLQGVTSWGYVP | |
| | CGTPNKPSVAVRVLSYVKWIEDTIAENS | |

In certain embodiments, a KLK1 polypeptide comprises, consists, or consists essentially of SEQ ID NO:1-3 or 4, or residues 1-262, residues 19-262, or residues 25-262 of SEQ ID NO:1 or SEQ ID NO:2, including fragments and variants thereof. Amino acids 1 to 18 of SEQ ID NO:1 and 2 represent the signal peptide, amino acids 19 to 24 represent propeptide sequences, and amino acids 25 to 262 represent the mature peptide. Thus, the preproprotein includes a presumptive 17-amino acid signal peptide, a 7-amino acid proenzyme fragment and a 238-amino acid mature KLK1 protein.

A comparison between SEQ ID NO:1 and SEQ ID NO:2 (or SEQ ID NO:3 and SEQ ID NO:4) shows two amino acid differences between the two hKLK1 amino acid sequences. Single-nucleotide polymorphism (SNPs) between the two individuals within a species account for an E to Q substitution at amino acid residue 145 of 262 and an A to V substitution at position 188 of 262. SEQ ID NO:1 has an E (glutamic acid) at position 145 and an A (alanine) at position 188, while SEQ ID NO:2 has a Q (glutamine) at position 145 and a V (valine) at position 188. In some embodiments, KLK1 polypeptide has an E at position 145; a Q at position 145; an A at position 188; an A at position 188; an E at position 145 and an A at position 188; a Q at position 145 and a V at position 188; a Q at position 145 and an A at position 188; or an E at position 145 and a V at position 188.

As noted above, certain embodiments include active variants and fragments of reference KLK1 polypeptide. A “variant” of a starting or reference polypeptide is a polypeptide that has an amino acid sequence different from that of the starting or reference polypeptide. Such variants include, for example, deletions from, insertions into, and/or substitutions of residues within the amino acid sequence of the polypeptide of interest. A variant amino acid, in this context, refers to an amino acid different from the amino acid at the corresponding position in a starting or reference polypeptide sequence. Any combination of deletion, insertion, and substitution may be made to arrive at the final variant or mutant construct, provided that the final construct possesses the desired functional characteristics. The amino acid changes also may alter post-translational processes of the polypeptide, such as changing the number or position of glycosylation sites.

In some embodiments, a KLK polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to a reference sequence, such as, for example, an amino acid sequence described herein (for example, SEQ ID NOs: 1-4).

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO:1 or 3, or to a fragment of SEQ ID NO:1 or 3, such as for example, residues 25-262 or residues 78-141 of SEQ ID NO:1. Such a KLK1 polypeptide may have an E or a Q at amino acid residue 145, and/or an A or a V at position 188.

In some aspects, a KLK1 polypeptide has at least about 80%, at least about 85%, at least about 90%, at least about 91%, at least about 92%, at least about 93%, at least about 94%, at least about 95%, at least about 96%, at least about 97%, at least about 98%, at least about 98.5%, at least about 99%, or at least about 99.5% amino acid identity to SEQ ID NO:2 or 4, or to a fragment of SEQ ID NO:2 or 4, such as for example, residues 25-262 or residues 78-141 of SEQ ID NO:2. Such a KLK1 polypeptide may have an E or a Q at amino acid residue 145, and/or an A or a V at position 188.

“Percent (%) amino acid sequence identity” with respect to a polypeptide is defined as the percentage of amino acid residues in a candidate sequence that are identical with the amino acid residues in the reference sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or Megalign (DNASTAR) software. Those skilled in the art can determine appropriate parameters for measuring alignment, including any algorithms needed to achieve maximal alignment over the full length of the sequences being compared. The ALIGN-2 program is publicly available through Genentech, Inc., South San Francisco, California.

For purposes herein, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) can be calculated as: 100 times the fraction X/Y, where X is the number of amino acid residues scored as identical matches by the sequence alignment program in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

Variants may also include heterologous sequences or chemical modifications which are added to the reference KLK1 polypeptide, for example, to facilitate purification, improve metabolic half-life, or make the polypeptide easier to identify. Examples include affinity tags such as a His-tag, Fc regions, and/or a PEGylation sequence and PEG.

The term “fragment” includes smaller portions of a KLK1 polypeptide (or variants thereof) that retain the activity of a KLK1 polypeptide. Fragments includes, for example, a KLK1 polypeptide fragment that ranges in size from about 20 to about 50, about 20 to about 100, about 20 to about 150, about 20 to about 200, or about 20 to about 250 amino acids in length. In other embodiments, a KLK1 polypeptide fragment ranges in size from about 50 to about 100, about 50 to about 150, about 50 to about 200, or about 50 to about 250 amino acids in length. In other embodiments, a KLK1 polypeptide fragment ranges in size from about 100 to about 150, about 100 to about 200, about 100 to about 250, about 150 to about 175, about 150 to about 200, or about 150 to about 250 amino acids in length. In other illustrative embodiments, a KLK1 polypeptide fragment ranges in size from about 200 to about 250 amino acids in length. Certain embodiments comprise a polypeptide fragment of a full-length KLK1 of about, up to about, or at least about 50, 60, 70, 80, 90, 100, 110, 120, 130, 140, 150, 160, 170, 180, 190, 200, 210, 220, 230, 240, 250 or more (e.g., contiguous) amino acid residues. In some embodiments, a fragment may have residues 25-262 or residues 78-141 of a preproprotein sequence. In some embodiments, a fragment may be any such fragment size, as described above, of SEQ D NO:1 or SEQ ID NO:2.

In some instances, fragments and variants of a KLK1 polypeptide retain the enzymatic capacity to release the vasoactive peptide, Lys-bradykinin, from low molecular weight kininogen. In some embodiments, an active variant or fragment retains serine protease activity of a KLK1 polypeptide that releases kallidin from a higher molecular weight precursor such as kininogen, or that cleaves a substrate similar to kininogen such as D-val-leu-arg-7 amido-4-trifluoromethylcoumarin to release a colorimetric or fluorometric fragment. The protease activity of KLK1 polypeptides can be measured in an enzyme activity assay by measuring either the cleavage of low-molecular-weight kininogen, or the generation of lys-bradykinin. In one assay format, a labeled substrate is reacted with the KLK1 glycoform, and the release of a labeled fragment is detected. One example of such a fluorogenic substrate suitable for KLK1 measurement of activity is D-val-leu-arg-7 amido-4-trifluoromethylcoumarin (D-VLR-AFC, FW 597.6) (Sigma, Cat #V2888 or Ana Spec Inc Cat #24137). When D-VLR-AFC is hydrolyzed, the free AFC produced in the reaction can be quantified by fluorometric detection (excitation 400 nm, emission 505 nm) or by spectrophotometric detection at 380 nm (extinction coefficient=12,600 at pH 7.2). Other methods and substrates may also be used to measure KLK1 proteolytic activity.

Glycoforms and Mixtures Thereof. In certain embodiments, a pharmaceutical composition or dosage form comprises a mixture of one or more KLK1 polypeptide glycoforms, including pharmaceutical compositions and dosage forms that comprise defined ratios of double and triple glycosylated KLK1 polypeptides (see U.S. application Ser. No. 14/677,122, incorporated by reference in its entirety).

Human kallikrein has three potential Asn-linked (N-linked) glycosylation sites at residues 78, 84, and 141, relative to the mature amino acid sequence shown, for example, in SEQ ID NO: 3 or 4, as well as putative O-linked glycosylation sites. However, O-linked glycosylation is not detected in naturally-occurring KLK1. By SDS-PAGE analysis, KLK1 polypeptides glycosylated at all three positions (positions 78, 84, and 141) are detected as the high molecular weight band and are referred to herein as the high-molecular weight, triple glycosylated glycoform of KLK1 (or “high glycoform” or “triple glycoform” KLK1). By SDS-PAGE analysis, KLK1 polypeptides glycosylated at only two of three available positions (positions 78 and 84) are detected as a low molecular weight band and are referred to herein as the low-molecular weight, double glycosylated glycoform of KLK1 (or as “low glycoform” or “double glycoform” KLK1).

Certain pharmaceutical compositions or dosage forms therefore comprise a mixture of KLK1 glycoforms at a defined ratio, for example, comprising a first KLK1 polypeptide and a second KLK1 polypeptide, wherein the first KLK1 polypeptide has three glycans attached at the three different positions available for glycosylation in the polypeptide, and wherein the second KLK1 polypeptide has two glycans attached at only two of the three different positions available for glycosylation in the polypeptide. In certain embodiments, the first and second KLK1 polypeptides are present in the pharmaceutical composition or dosage form at a ratio of about 45:55 to about 55:45, including, for example, about 46:54, about 47:53, about 48:52, about 49:51, about 51:49, about 52:48, about 53:47, and about 54:46, including all integers and decimal points in between. In specific embodiments, the first and second KLK1 polypeptides are present in the pharmaceutical composition or dosage form at a ratio of about 50:50. In some embodiments, the ratio of the first and second KLK1 polypeptides is not about 60:40. In some embodiments, the ratio of the first and second KLK1 polypeptides is not about 40:60. In certain embodiments, the pharmaceutical composition or dosage form is free or substantially free of other glycosylated isoforms (glycoforms) of KLK1.

Some pharmaceutical compositions or dosage forms comprise a triple glycoform of a KLK1 polypeptide and a double glycoform of a KLK1 polypeptide, wherein the triple glycoform and the double glycoform are present in the pharmaceutical composition or dosage form at a ratio of about 45:55 to about 55:45 including, for example, about 46:54, about 47:53, about 48:52, about 49:51, about 51:49, about 52:48, about 53:47, and about 54:46. In some embodiments, the triple glycoform and the double glycoform are present in the pharmaceutical composition or dosage form at a ratio of about 50:50. In some embodiments, the ratio of the triple glycoform and double glycoform is not about 60:40. In some embodiments, the ratio of the triple glycoform and double glycoform is not about 40:60. In certain embodiments, the pharmaceutical composition or dosage form is free or substantially free of other glycosylated isoforms (glycoforms) of KLK1.

In certain embodiments, the pharmaceutical composition or dosage form comprises DM199. “DM199” refers to a formulation composed of two glycoforms of a mature, human KLK1 variant polypeptide, each glycoform having the amino acid sequence set forth in SEQ ID NO: 4 (also amino acid residues 25-262 of SEQ ID NO: 2): one being a triple glycoform that has three N-linked glycans attached at residues 78, 84, and 141, and the other being a double glycoform that has two N-linked glycans attached at residues 78 and 84 but not 141 (the numbering being defined by SEQ ID NO: SEQ ID NO: 3 or 4), wherein the triple glycoform and the double glycoform are formulated at a ratio of about 50:50.

The ratios of the double and triple glycosylated isoforms of KLK1 can be detected and quantitated by a variety of methods, including high performance liquid chromatography (HPLC), which may include reversed phase (RP-HPLC), lectin affinity chromatography and lectin affinity electrophoresis. The preparation and characterization of KLK1 glycoform mixtures is described in U.S. application Ser. No. 14/677,122, incorporated by reference in its entirety.

Additional Agents. In certain embodiments, a pharmaceutical composition or dosage form comprises one more additional therapeutic agents, for example, a second therapeutic agent. In some embodiments, the additional agent is selected from one or more of an angiotensin receptor blocker, edavarone, finerenone, and bardoxalone, including combinations thereof. Examples of angiotensin receptor blockers include losartan, azilsartan, candesartan, eprosartan, fimasartan, irbesartan, olmesartan, saprisartan, telmisartan, and valsartan, including combinations thereof.

Purity. In some embodiments, the “purity” of a pharmaceutical composition or dosage form is characterized, for example, by the amount (e.g., total amount, relative amount, percentage) of host cell protein(s), host cell DNA, endotoxin, and/or percentage single peak purity by SEC HPLC. In some instances, the purity of a pharmaceutical composition or dosage form is characterized by the amount (e.g., percentage) of KLK1 polypeptide relative to other components, for example, any one or more of the foregoing.

In some embodiments, the purity of a pharmaceutical composition or dosage form is characterized relative to or by the levels or amount of host cell proteins. The host cells used for recombinant expression may range from bacteria and yeast to cell lines derived from mammalian or insect species. The cells contain hundreds to thousands of host cell proteins (HCPs) and other biomolecules that could contaminate the final product. The HCP may be secreted along with the protein of interest, or released by accidental lysing of the cells, and may contaminate the protein of interest. Two types of immunological methods may be applied to HCP analysis: Western blotting (WB) and immunoassay (IA), which includes techniques such as ELISA and sandwich immunoassay or similar methods using radioactive, luminescent, or fluorescent reporting labels. Compositions of the present invention may include host cell protein of less than about 500, less than about 400, less than about 300, less than about 200, less than about 100 or less than about 50 ng/mg total protein.

In some instances, purity is characterized relative to or by the levels or amount of host cell DNA. Detection of residual host cell DNA may be performed by Polymerase Chain Reaction (PCR) with a variety of primers for sequences in the host cell genome. Residual host cell DNA is generally reported as being below a certain threshold level, but may also be quantitated with a rPCR method. Compositions of the present invention may include host cell deoxyribonucleic acid (DNA) of less than about 100, less than about 90, less than about 80, less than about 70, less than about 60, less than about 50, less than about 40, less than about 30, less than about 20, or less than about 10 pg/mg total protein.

In certain embodiments, purity is characterized relative to or by the amount or levels of endotoxin. As noted herein, endotoxin is extremely potent, heat stable, passes sterilizing membrane filters, and is present everywhere bacteria are or have been present. An Endotoxin Unit (EU) is a unit of biological activity of the USP Reference Endotoxin Standard.

The bacterial endotoxins test (BET) is a test to detect or quantify endotoxins from Gram-negative bacteria using amoebocyte lysate (white blood cells) from the horseshoe crab (Limulus polyphemus or Tachypleus tridentatus). Limulus amoebocyte lysate (LAL) reagent, FDA approved, is used for all USP endotoxin tests. There are at least three methods for this test: Method A, the gel-clot technique, which is based on gel formation; Method B, the turbidimetric technique, based on the development of turbidity after cleavage of an endogenous substrate; and Method C, the chromogenic technique, based on the development of color after cleavage of a synthetic peptide-chromogen complex.

At least two types of endotoxin tests are described in the USP <85> BET. Photometric tests require a spectrophotometer, endotoxin-specific software and printout capability. The simplest photometric system is a handheld unit employing a single-use LAL cartridge that contains dried, pre-calibrated reagents; there is no need for liquid reagents or standards. The FDA-approved unit is marketed under the name of Endosafe®-PTS™. The device requires about 15 minutes to analyze small amounts of sample, a 25 μL aliquot from CSP diluted in a sterile tube, and to print out results. In contrast, gel-clot methods require a dry-heat block, calibrated pipettes and thermometer, vortex mixer, freeze-dried LAL reagents, LAL Reagent Water (LRW) for hydrating reagents and depyrogenated glassware. In this clot test, diluted sample and liquid reagents require about an hour for sample and positive-control preparation and an hour's incubation in a heat block; results are recorded manually. Thus, the simplicity and speed of the automated system make it ideally suited to the pharmacy setting.

In some instances, the purity of a pharmaceutical composition or dosage form is characterized by the degree of aggregation. For instance, the degree of aggregation of KLK1 can be determined by Size-exclusion chromatography (SEC), which separates particles on the basis of size. It is a generally accepted method for determining the tertiary structure and quaternary structure of purified proteins. SEC is used primarily for the analysis of large molecules such as proteins or polymers. SEC works by trapping these smaller molecules in the pores of a particle. The larger molecules simply pass by the pores as they are too large to enter the pores. Larger molecules therefore flow through the column quicker than smaller molecules, that is, the smaller the molecule, the longer the retention time. Certain compositions are also substantially free of aggregates (greater than about 95% appearing as a single peak by SEC HPLC). Certain embodiments are free of aggregates with greater than about 96%, about 97%, about 98%, or about 99%, appearing as a single peak by SEC HPLC.

In certain embodiments, the “purity” of the KLK1 polypeptide(s) in a pharmaceutical composition or dosage form is specifically defined. For instance, certain pharmaceutical compositions or dosage forms comprise one or more hKLK1 polypeptides that are at least about 80, at least about 85, at least about 90, at least about 91, at least about 92, at least about 93, at least about 94, at least about 95, at least about 96, at least about 97, at least about 98, at least about 99, or 100% pure, including all decimals in between, relative to other components in the pharmaceutical composition or dosage form. Purity can be measured, for example and by no means limiting, by high performance liquid chromatography (HPLC), a well-known form of column chromatography used frequently in biochemistry and analytical chemistry to separate, identify, and quantify compounds.

In certain embodiments, a pharmaceutical composition or dosage form has one or more of the following determinations of purity: less than about 1 EU endotoxin/mg protein, less that about 100 ng host cell protein/mg protein, less than about 10 pg host cell DNA/mg protein, and/or greater than about 95% single peak purity by SEC HPLC.

In some instances, a pharmaceutical composition or dosage form is formulated with pharmaceutically acceptable excipients, diluents, adjuvants, or carriers, for instance, to optimize stability and achieve isotonicity. In certain aspects, the pH of the pharmaceutical composition or dosage form is near physiological pH or about pH 7.4, including about pH 6.5, about 7.0, about 7.1, about 7.2, about 7.3, about 7.4, about 7.5, about 7.6, about 7.7, about 7.8, about 7.9, about 8.0, about 8.5, or any range thereof. In some embodiments, a pharmaceutical composition or dosage form comprises a KLK1 polypeptide in combination with a physiologically acceptable carrier. Such carriers include pharmaceutically acceptable carriers, excipients, or stabilizers which are nontoxic to the cell or mammal being exposed thereto at the dosages and concentrations employed. Methods of formulation are well known in the art and are disclosed, for example, in Remington: The Science and Practice of Pharmacy, Mack Publishing Company, Easton, Pa., Edition 21 (2005).

The phrase “physiologically-acceptable” or “pharmaceutically-acceptable” refers to molecular entities and compositions that do not produce a significant allergic or similar untoward reaction when administered to a human. Typically, such compositions are prepared as injectables, either as liquid solutions or suspensions; solid forms suitable for solution in, or suspension in, liquid prior to injection can also be prepared. The preparations can also be emulsified.

As used herein, “carrier” includes any and all solvents, dispersion media, vehicles, coatings, diluents, isotonic and absorption delaying agents, buffers, carrier solutions, suspensions, colloids, and the like. Except insofar as any conventional media or agent is incompatible with the active ingredient, its use in the therapeutic compositions is contemplated.

The pharmaceutical compositions and dosage forms described herein may be formulated for administered by a variety of techniques, including, for example, subcutaneous and intravenous administration. Particular embodiments include administration by subcutaneous injection. In some instances, a subcutaneous injection (abbreviated as SC, SQ, sub-cu, sub-Q or subcut with SQ) is administered as a bolus into the subcutis, the layer of skin directly below the dermis and epidermis, collectively referred to as the cutis. Exemplary places on the body where people can inject SC most easily include, without limitation, the outer area of the upper arm, just above and below the waist, excepting in certain aspects the area right around the navel (a˜2-inch circle), the upper area of the buttock, just behind the hip bone, and the front of the thigh, midway to the outer side, about 4 inches below the top of the thigh to about 4 inches above the knee. These areas can vary with the size of the person. Also, changing the injection site can prevent lumps or small dents called lipodystrophies from forming in the skin.

Subcutaneous injections usually go into the fatty tissue below the skin and in certain instances can utilize a smaller, shorter needle. In specific instances, a needle that is about ½ inch to about ⅝ of an inch in length with a gauge of about 25 to about 31 is sufficient to subcutaneously administer the medication. As will be appreciated by someone skilled in the art, these are general recommendations and SC injections may be administered with needles of other sizes. In some embodiments SC administration is performed by pinching-up on the tissue to prevent injection into the muscle, and/or insertion of the needle at a ˜45° angle to the skin.

In certain instances, administration of the pharmaceutical composition or dosage form achieves in the subject a therapeutically-effective serum level of the one or more KLK1 polypeptides. In some instances, administration of the pharmaceutical composition or dosage form achieves a therapeutically-effective serum level of the one or more KLK1 polypeptides in about or less than about 0.5, 1, 2, 3, 4, 5, 6, 7, 8, 9, 10, 11, 12, 13, 14, 15, 16, 17, 18, 19, or 20 hours following administration. In some instances, the pharmaceutical composition or dosage form is administered intravenously or subcutaneously. In some instances, the therapeutically-effective serum level is about or at least about 1.0 to about or at least about 5.0 ng/ml, or about or at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/ml, including all ranges in between.

In some instances, administration of the pharmaceutical composition or dosage form achieves and maintains in the subject a therapeutically-effective serum level of the one or more KLK1 polypeptides. For instance, in some embodiments, administration of the pharmaceutical composition or dosage forms achieves a therapeutically-effective serum level of the one or more KLK1 polypeptides in about or less than about 0.5, 1, 2, 3, 4, 5, 6, 7, 8, 9, 10, 11, 12, 13, 14, 15, 16, 17, 18, 19, or 20 hours, and maintains in the subject a therapeutically-effective serum level of the one or more KLK1 polypeptides for about or at least about 0.5, 1, 2, 4, 6, 8, 10, 12, 24, 23, 48, 60, 72, 84, 96 hours or more, or 1, 2, 3, 4, 5, 6, 7, 8, 9, 10, 11, 12, 13, or 14 days or more, following the administration (e.g., a single subcutaneous or intravenous administration). In some instances, the therapeutically-effective serum level is about or at least about 1.0 to about or at least about 5.0 ng/ml, or about or at least about 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, or 5.0 mg/ml, including all ranges in between.

Certain embodiments include a dosage regimen of administering one or more KLK1 pharmaceutical compositions or dosage forms at defined intervals over a period of time. For example, certain dosage regimens include administering a KLK1 pharmaceutical composition or dosage form once or twice a day, once or twice every two days (e.g., once a day every other day), once or twice every three days (e.g., once a day every third day following an initial or earlier administration), once or twice every four days, once or twice every five days, once or twice every six days, once or twice every week, once or twice every other week. Specific dosage regimens include administering a KLK1 pharmaceutical composition or dosage form once a day every three days (e.g., once a day every third day following an initial or earlier administration), including wherein the pharmaceutical composition or dosage form is administered subcutaneously.

Specific embodiments include intravenously administering at least one intravenous pharmaceutical composition or dosage form to the subject, followed by subcutaneously administering one or more subcutaneous dosages form to the subject, for example, as a dosing regimen of about once or twice a day, once or twice every two days, once or twice every three days, once or twice every four days, once or twice every five days, once or twice every six days, once or twice every week. In particular embodiments, the intravenous administration of the pharmaceutical composition or dosage form achieves in the subject a therapeutically-effective serum level of the one or more KLK1 polypeptides in about or less than about 0.5, 1, 2, 3, or 4 hours following the intravenous administration, and the subcutaneous administration of the pharmaceutical composition or dosage form maintains the therapeutically-effective serum level for about or at least about 2, 4, 6, 8, 10, 12, 24, 23, 48, 60, 72, 84, 96 hours or more, or 1, 2, 3, 4, 5, 6, 7, 8, 9, 10, 11, 12, 13, or 14 days or more, following the subcutaneous administration.

Some variation in dosage will necessarily occur depending on the condition of the subject being treated. The person responsible for administration will, in any event, determine the appropriate dose for the individual subject. Moreover, for human administration, preparations should meet sterility, pyrogenicity, and general safety and purity standards as required by the FDA. In some instances, preparation are substantially endotoxin-free or pyrogen-free, as described herein. According to the FDA Guidance for Industry; Estimating the Maximum Safe Starting Dose in Initial Clinical Trial for Therapeutics in Adult Healthy Volunteers (July 2005), Appendix D: Converting animal doses to human equivalent doses. A human equivalent dose is 1/7 the rat dose and a human equivalent dose is 1/12 a mouse dose.

In some embodiments, a pharmaceutical composition or dosage form described herein is administered with one or more additional therapeutic agents or modalities. In some aspects, administration of the pharmaceutical composition or dosage form allows for the effectiveness of a lower dosage of other therapeutic modalities when compared to the administration of the other therapeutic modalities alone, providing relief from the toxicity observed with the administration of higher doses of the other modalities. One or more additional therapeutic agents may be administered before, after, and/or coincident (e.g., together with) to the administration of a pharmaceutical composition or dosage form described herein. A pharmaceutical composition or dosage form and any additional therapeutic agents can be administered separately or as part of the same mixture or cocktail. As used herein, an additional therapeutic agent includes, for example, an agent whose use for the treatment of a condition (e.g., an ischemic or hemorrhagic condition) is known to persons skilled in the art. Examples of additional agents include angiotensin receptor blockers, edavarone, finerenone, and bardoxalone, including combinations thereof. Particular examples of angiotensin receptor blockers include losartan, azilsartan, candesartan, eprosartan, fimasartan, irbesartan, olmesartan, saprisartan, telmisartan, and valsartan, including combinations thereof.

In some embodiments, administering the pharmaceutical composition improves one or more clinical parameters in the patient. In certain embodiments, the one or more clinical parameters are selected from decreased albuminuria (UACR), increased estimated glomerular filtration rate (eGFR), decreased blood pressure, serum KLK1 levels of about 1-5 ng/ml, decreased swelling, including in the lower extremities of the patient, and decreased risk or occurrence of cardiovascular events in the patient, including decreased risk or occurrence of myocardial infarction or stroke. In specific embodiments, administering the pharmaceutical composition decreases UACR by about or at least about 25, 30, 35, 40, 45, 50, 55, 60, 65, or 70% or more. Any one or more of the foregoing clinical parameters can be measured according to routine clinical techniques in the art.

Devices. Also included are devices that comprise a pharmaceutical composition or dosage form described herein, including devices suitable for subcutaneous or intravenous delivery, and related methods of use thereof. In some embodiments, the device is a syringe. In some embodiments, the syringe is attached to a hypodermic needle assembly, optionally comprising a protective cover around the needle assembly. In some embodiments, the needle may be about ½ inch to about ⅝ of an inch in length and has a gauge of about 25 to about 31. Certain embodiments thus include devices that attached or attachable to a needle assembly that is suitable for subcutaneous administration, comprising a pharmaceutical composition or dosage form described herein. For example, certain devices include a vial or syringe, optionally where the vial or syringe is attachable to or is attached to a hypodermic needle assembly. Also included are vials having a rubber cap, where a needle/syringe can be inserted into the vial via the rubber cap to withdraw the pharmaceutical composition or dosage form for subcutaneous administration.

In particular aspects, the device is a syringe that is attachable or attached to a hypodermic needle, and is packaged with one or more removable and/or permanent protective covers around the needle or needle assembly. For instance, a first removable protective cover (which is removed during administration) can protect a user or other person from the needle prior to administration, and a second protective cover can be put (i.e., snapped) into place for safe disposal of the device after administration.

The present invention is illustrated by the following examples. It is to be understood that the particular examples, materials, amounts, and procedures are to be interpreted broadly in accordance with the scope and spirit of the invention as set forth herein.

### Example 1

### Urinary KLK1 Levels in Patients with CKD Relative to Healthy Subjects

Analyses were performed to evaluate differences in urinary KLK1 between healthy patients and patients with CKD.

Description of the CKD patient population: Male and female greater than 18 years of age with CKD were defined by using CKD-EPI for Stage II (60 to <90 mL/min/1.73 m²) or Stage III (30 to <60 mL/min/1.73 m²). Patients must have had UACR levels >150 mg/g and <5000 mg/g at screening. Analysis of KLK1 levels was performed using an electro-chemiluminescent assay validated by Kansas City Bio.

Description of healthy patient population: Twenty normal (healthy) volunteers, ranging from 23 to 66 years of age, provided urine samples for analysis of KLK1 levels using an electro-chemiluminescent assay validated by Kansas City Bio. The population included both males and females from various ethnicities, including Hispanic, Black, and Caucasian. All donors were confirmed to not have been diagnosed with cancer, HIV, or liver diseases.

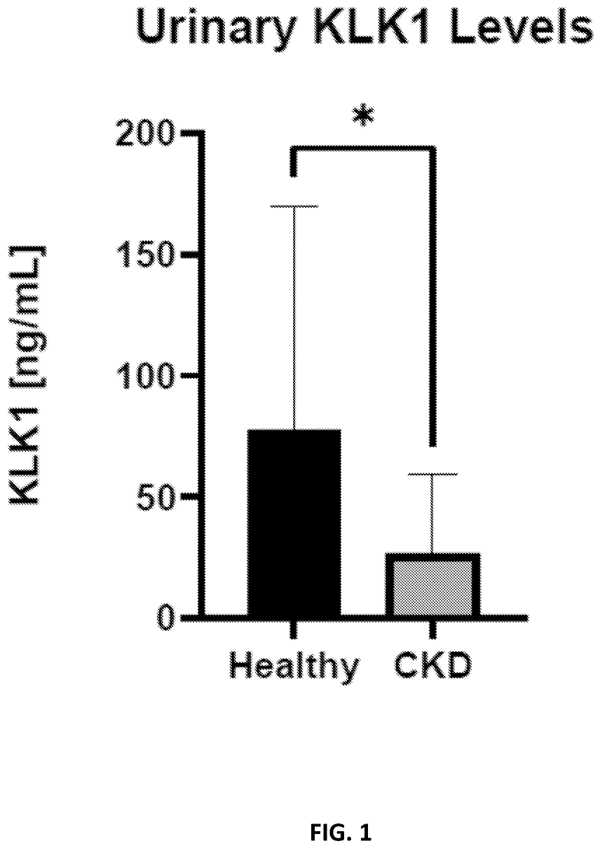

Data Analysis (see FIG. 1): Data were analyzed in GraphPad Prism 9.3.1. The urine KLK1 levels for healthy and CKD patients were compared using an unpaired t-test. As the data were not normally distributed, a Mann-Whitney analysis was used. Results show a significant (p=0.0131) difference in urine KLK1 levels between healthy patients and those with CKD, evidencing that urinary KLK1 levels can be used to identify optimal patient populations that will most benefit from KLK1 therapy.

Data analysis (see Table E1): Data were analyzed in GraphPad Prism 9.3.1 using an unpaired t-test. As the data were not normally distributed, a Mann-Whitney analysis was used. For subjects in both groups with KLK1 levels below the limit of quantitation (BLOQ), their values were replaced with the LOQ/sqrt(2). The LOQ is 10 ng/mL, so BLOQ values thus became 10/1.41=7.07 ng/mL. In a complete case analysis (CCA) where BLOQ levels were discarded, significance between groups was still achieved (p=0.0073). Based on the patient data, at least 80% of CKD patients will be below the 50^{th} percentile of the healthy population.

TABLE E1
Analysis of Urinary KLK1 Levels

| Percentile | Urinary KLK1 [ng/mL] |
|---|---|
| 75^{th} | 119.3 |
| 60^{th} | 69.7 |
| 50^{th} | 39.4 |
| 40^{th} | 23.8 |
| 30^{th} | 12.9 |
| 25^{th} | 8.5 |
| 20^{th} | 7.1 |

The urinary KLK1 levels of the 50^{th} percentile and below thus represent a strategy for identifying optimal CKD patient populations that will most benefit from KLK1 therapy.

1. A method of treating chronic kidney disease (CKD) in a patient in need thereof, comprising administering to the patient a pharmaceutical composition that comprises one or more tissue kallikrein (KLK1) polypeptides, wherein the patient has low KLK1 levels and/or salt-sensitive hypertension.

2. The method of claim 1, wherein the low KLK1 levels are characterized by urinary KLK1 levels of about or less than about 15, 16, 17, 18, 19, 20, 25, 30, 35, or 40 ng/mL.

3. The method of claim 1, comprising:
determining KLK1 levels in a urine or blood/serum sample from the patient; and
administering the pharmaceutical composition to the patient if urinary KLK1 levels are about or less than about 15, 16, 17, 18, 19, 20, 25, 30, 35, or 40 ng/mL.

4. The method of claim 1, wherein the patient has:
an R53H mutation in exon 3 of the KLK1 gene;
a 12G promoter allele in the KLK1 gene, which is characterized by 12 G repeats in the KLK1 gene locus starting at position −130 and ending at position −121;
an APOL1 gene mutation of the G1 haplotype, which is characterized by a terminal exon with two SNPs: rs73885319 and rs609101, and/or the G2 haplotype, which is characterized by a six base pair deletion: rs71785313;
a T594M mutation in the epithelial sodium-channel beta subunit (ENaC) gene;
a CYP11B1 gene mutation, characterized by an rs6410 single nucleotide polymorphism (SNP) and/or an rs6387 SNP;
a CYP11B2 gene mutation, which is characterized by an intron 2 conversion, an rs1799998 SNP, and/or an rs4539 SNP;
a SLC12A1 gene mutation, which is characterized by a base pair deletion, an insertion, and/or a nonconservative missense mutation in the SLC12A1 gene; and/or
a V142I mutation in the transthyretin (TTR) gene.

5. The method of claim 4, comprising:
determining R53H mutation status in exon 3 of the KLK1 gene in a biological sample from the patient; and
administering the pharmaceutical composition to the patient if the R53H mutation in exon 3 of the KLK1 gene is present in the biological sample, optionally if the R53H mutation is homozygous.

6. The method of claim 4, comprising:
determining 12G promoter allele status in the KLK1 gene in a biological sample from the patient; and
administering the pharmaceutical composition to the patient if the 12G promoter allele in the KLK1 gene is present in the biological sample, optionally if the 12G promoter allele is homozygous.

7. The method of claim 4, comprising:
determining APOL1 gene mutation status in a biological sample from the patient; and
administering the pharmaceutical composition to the patient if the APOL1 gene mutation is present in the biological sample as at least one of the G1 haplotype and/or the G2 haplotype, optionally if the APOL1 gene mutation is homozygous.

8. The method of claim 4, comprising:
determining T594M mutation status in the ENaC gene in a biological sample from the patient; and
administering the pharmaceutical composition to the patient if the T594M mutation in the ENaC gene is present in the biological sample, optionally if the T594M mutation is homozygous.

9. The method of claim 4, comprising:
determining CYP11B1 gene mutation status in a biological sample from the patient; and
administering the pharmaceutical composition to the patient if the CYP11B1 gene mutation is present in the biological sample, optionally if the CYP11B1 gene mutation is homozygous.

10. The method of claim 4, comprising:
determining CYP11B2 gene mutation status in a biological sample from the patient; and
administering the pharmaceutical composition to the patient if the CYP11B2 gene mutation is present in the biological sample, optionally if the CYP11B2 gene mutation is homozygous.

11. The method of claim 4, comprising:
determining SLC12A1 gene mutation status in a biological sample from the patient; and
administering the pharmaceutical composition to the patient if the SLC12A1 gene mutation is present in the biological sample, optionally if the SLC12A1 gene mutation is homozygous.

12. The method of claim 4, comprising:
determining V142I mutation status in in the TTR gene in a biological sample from the patient; and
administering the pharmaceutical composition to the patient if the V142I mutation in the TTR gene is present in the biological sample, optionally if the V142I mutation is homozygous.

13. (canceled)

14. The method of claim 1, wherein the patient is of African, Asian, Spanish, or Polynesian descent, optionally an African-American.

15. The method of claim 1, wherein the patient has sickle cell disease and/or focal segmental glomerulosclerosis (FSGS).

16. The method of claim 1, comprising obtaining the biological sample from the patient.

17. The method of claim 1, wherein the biological sample is a blood/serum sample or a urine sample.

18. The method of claim 1, wherein the pharmaceutical composition comprises DM199.

19. The method of claim 1, wherein the pharmaceutical composition comprises a first KLK1 polypeptide and a second KLK1 polypeptide, wherein the first KLK1 polypeptide has three N-linked glycans attached at three different positions per polypeptide and the second KLK1 polypeptide has two N-linked glycans attached at two different positions per polypeptide, wherein the three glycans of the first KLK1 polypeptide are N-linked glycans at residues 78, 84, and 141, and wherein the two glycans of the second KLK1 polypeptide are N-linked glycans at residues 78 and 84 but not 141, and wherein the first KLK1 polypeptide and the second KLK1 polypeptides are present in the pharmaceutical composition at a ratio of about 45:55 to about 55:45.

20-23. (canceled)

24. The method of claim 19, wherein the first KLK1 polypeptide and the second KLK1 polypeptides are present in the pharmaceutical composition at a ratio of about 50:50.

25. The method of claim 1, wherein the one or more KLK1 polypeptide(s) are recombinant KLK polypeptides, mature KLK1 polypeptides, human KLK1 (hKLK1) polypeptides, or any combination thereof.

26. The method of claim 25, wherein the hKLK1 polypeptide(s) comprise, consist, or consist essentially of amino acid residues 78-141 of SEQ ID NO:1 or amino acids residues 78-141 SEQ ID NO:2, or an active fragment thereof, or an active variant having at least about 90, 95, 96, 97, 98, or 99% sequence identity to amino acid residues 78-141 of SEQ ID NO:1 or amino acids residues 78-141 SEQ ID NO:2.

27. The method of claim 25, wherein the hKLK1 polypeptide(s) comprise, consist, or consist essentially of amino acid residues 25-262 of SEQ ID NO:1 or amino acid residues 25-262 of SEQ ID NO:2, or an active fragment thereof, or an active variant having at least about 90, 95, 96, 97, 98, or 99% sequence identity to amino acid residues 25-262 of SEQ ID NO:1 or amino acid residues 25-262 of SEQ ID NO:2.

28. The method of claim 25, wherein the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises E145 and/or A188.

29. The method of claim 25, wherein the KLK1 polypeptide(s) comprise an amino acid sequence having at least about 90, 95, 96, 97, 98, or 99% sequence identity to amino acid residues 25-262 of SEQ ID NO:2, and wherein the KLK1 polypeptide(s) comprises Q145 and/or V188.

30. The method of claim 1, wherein the pharmaceutical composition is formulated at a total KLK1 polypeptide dosage of about 0.5 μg/kg to about 10.0 μg/kg.

31. The method of claim 30, wherein the pharmaceutical composition is formulated at a total KLK1 polypeptide dosage of about 2 μg/kg or about 4 μg/kg, or about 0.1, 0.3, 0.4, 0.5, 0.6, 0.7, 0.75, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, or 10 μg/kg.

32. The method of claim 30, wherein the pharmaceutical composition is formulated at a total KLK1 polypeptide dosage of about 1.0 μg/kg to about 5.0 μg/kg, or about 1.0 μg/kg to about 4.0 μg/kg, or about 1.0 μg/kg to about 3.0 μg/kg, or about 1.0 μg/kg to about 2.0 μg/kg, or about 2.0 μg/kg to about 5.0 μg/kg, or about 2.0 μg/kg to about 4.0 μg/kg, or about 2.0 μg/kg to about 3.0 μg/kg, or about 3.0 μg/kg to about 5.0 μg/kg, or about 3.0 μg/kg to about 4.0 μg/kg, or about 4.0 μg/kg to about 5.0 μg/kg.

33. The method of claim 1, comprising subcutaneously or intravenously administering the pharmaceutical composition to the patient.

34-39. (canceled)


SEQUENCE LISTING (4 sequences)

SEQ ID NO: 1 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 2 - protein, 262 aa, Homo sapiens
MWFLVLCLALSLGGTGAAPPIQSRIVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS

SEQ ID NO: 3 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTEEPEVGSTCLASGWGSIEPENFSFPDDLQCVDLKILPNDECKKAHVQKVTDFMLCVGHLEGGKDTCVGDSGGPLMCDGVLQGVTSWGYVPCGTPNKPSVAVRVLSYVKWIEDTIAENS

SEQ ID NO: 4 - protein, 238 aa, Homo sapiens
IVGGWECEQHSQPWQAALYHFSTFQCGGILVHRQWVLTAAHCISDNYQLWLGRHNLFDDENTAQFVHVSESFPHPGFNMSLLENHTRQADEDYSHDLMLLRLTEPADTITDAVKVVELPTQEPEVGSTCLASGWGSIEPENFSFPDDLQCVDLKILPNDECKKVHVQKVTDFMLCVGHLEGGKDTCVGDSGGPLMCDGVLQGVTSWGYVPCGTPNKPSVAVRVLSYVKWIEDTIAENS